(19) United States
(12) Patent Application Publication
(10) Pub. No.: US 2022/0305239 A1
(43) Pub. Date: Sep. 29, 2022

(54) Apparatus with Double Balloon for Treating Trigeminal Neuralgia

(71) Applicant: DIXI Neurolab, Inc., Oxford, MI (US)

(72) Inventor: John Thomas Gale, Myrtle Beach, SC (US)

(21) Appl. No.: 17/656,483

(22) Filed: Mar. 25, 2022

Related U.S. Application Data
(60) Provisional application No. 63/166,401, filed on Mar. 26, 2021.

(51) Int. Cl. A61M 25/10; A61B 18/14

(52) U.S. Cl. CPC A61M 25/1011; A61B 18/1492; A61B 2017/003

(57) ABSTRACT

An apparatus for performing a medical procedure includes a shaft having proximal and distal ends, a double balloon attached to the distal end of the shaft, and one or more fluid lines disposed in the shaft. The double balloon includes a first balloon, a second balloon, and a bridge extending between the first and second balloons. The fluid line(s) are fluidly coupled to the double balloon and configured to change an inflation state of the double balloon.

### CROSS-REFERENCE TO RELATED APPLICATIONS

This application claims priority to U.S. Provisional Application No. 63/166,401, titled “Apparatus With Double Balloon For Treating Trigeminal Neuralgia,” filed on Mar. 26, 2021, which is hereby incorporated by reference.

### TECHNICAL FIELD

This application relates generally to medical devices, such as those for treating trigeminal neuralgia.

### BACKGROUND

The trigeminal nerve, or fifth cranial nerve, is used to transmit sensory and motor information to the brain. It includes three major branches. The upper (ophthalmic or “V1”) branch connects the scalp, forehead, and front of head to the brain. The middle (maxillary or “V2”) branch connects the cheeks, upper jaw, top lip, teeth, gums, and side of nose to the brain. The lower (mandibular or “V3”) branch connects the lower jaw, teeth, gums, and bottom lip to the brain. The upper and middle branches only provide sensory information while the lower branch provides sensory information and motor functionality. The major branches are joined at the trigeminal ganglion 100, as illustrated in FIG. 1, which is located in Meckel's cave.

Trigeminal neuralgia (a/k/a tic douloureux) is a chronic neuropathic pain condition affecting the trigeminal nerve. Typical (Type 1) trigeminal neuralgia causes extreme, sporadic, and sudden burning or shock-like pain on one side of the face that can last from seconds to a few minutes and may occur in quick succession. Typical trigeminal neuralgia can be triggered by vibration of or contact with the face, such as when washing the face, shaving, eating, drinking, or talking. The typical condition can be progressive and worsen over time with fewer and shorter pain-free periods. Atypical (Type 2) trigeminal neuralgia causes constant burning or pain that is less intensive than in typical trigeminal neuralgia.

Current treatment includes medication and/or surgery. Medications can include anticonvulsants to block nerve firing or tricyclic antidepressants to treat pain. Surgical treatment can include a rhizotomy, the intentional damaging of nerve fibers to block pain.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

An aspect of the invention is directed to an apparatus comprising: a shaft having proximal and distal ends; a double balloon attached to the distal end of the shaft; and one or more fluid lines disposed in the shaft, the fluid line(s) fluidly coupled to the double balloon and configured to change an inflation state of the double balloon. The double balloon comprises a first balloon; a second balloon; and a bridge extending between the first and second balloons.

In one or more embodiments, the one or more fluid lines include: a first fluid line fluidly coupled to the first balloon, and a second fluid line fluidly coupled to the second balloon. In one or more embodiments, the first balloon and the second balloon are fluidly isolated from each other, whereby an inflation state of the first balloon is independent from an inflation state of the second balloon. In one or more embodiments, the first balloon has a first cavity to receive fluid from the first fluid line, the second balloon has a second cavity to receive fluid from the second fluid line, and the apparatus further comprises: a first pressure sensor in fluid communication with first cavity; and a second pressure sensor in fluid communication with second cavity.

In one or more embodiments, the bridge comprises a bridge balloon having a bridge inflation state, and the bridge balloon is fluidly coupled to the first balloon or the second balloon, whereby: when the bridge balloon is fluidly coupled to the first balloon, the bridge inflation state is the same as the first inflation state, and when the bridge balloon is fluidly coupled to the second balloon, the bridge inflation state is the same as the second inflation state. In one or more embodiments, the bridge comprises a bridge balloon, the one or more fluid lines include a third fluid line fluidly coupled to the bridge balloon, the first balloon, the second balloon, and the third balloon are fluidly isolated from each other, whereby an inflation state of the first balloon, an inflation state of the second balloon, and an inflation state of the bridge balloon are independent from each other.

In one or more embodiments, when the first and second balloons are inflated, a first dimension of the first balloon is larger than a corresponding second dimension of the second balloon. In one or more embodiments, when the first and second balloons are inflated, the first balloon has a larger diameter than the second balloon. In one or more embodiments, the second balloon is located between the bridge and the shaft.

In one or more embodiments, the apparatus further comprises an electrode contact arm attached to the shaft, the electrode arm including a plurality of electrical contacts. In one or more embodiments, the apparatus further comprises one or more wires mechanically coupled to the electrode arm to steer the electrode arm independently of the shaft.

In one or more embodiments, the apparatus further comprises an injectrode attached to the shaft. In one or more embodiments, the injectrode includes: a lumen to deliver a therapeutic agent; and one or more electrodes disposed on the lumen. In one or more embodiments, the apparatus further comprises one or more wires mechanically coupled to the lumen to steer the injectrode independently of the shaft.

Another aspect of the invention is directed to a system comprising: a shaft having proximal and distal ends; a double balloon attached to the distal end of the shaft, one or more fluid lines disposed in the shaft, the fluid line(s) fluidly coupled to the double balloon and configured to change an inflation state of the double balloon; and a fluid reservoir fluidly coupled to the one or more fluid lines. The double balloon comprises: a first balloon; a second balloon; and a bridge extending between the first and second balloons.

In one or more embodiments, the system further comprises a pressure sensor configured to measure a pressure of the one or more fluid lines. In one or more embodiments, the system further comprises a pump fluidly coupled to the fluid reservoir; and a controller in electrical communication with the pressure sensor and the pump, the controller configured to control the pump to inflate the first and second balloons while using the pressure of the one or more fluid lines as feedback.

Another aspect of the invention is directed to a method of performing a therapeutic procedure, the method comprising: inserting an apparatus into a ventricular space of a patient, the apparatus including: a shaft having proximal and distal ends; a double balloon attached to the distal end of the shaft, one or more fluid lines disposed in the shaft, the fluid line(s) fluidly coupled to the double balloon and configured to change an inflation state of the double balloon. The double balloon comprises: a first balloon; a second balloon; and a bridge extending between the first and second balloons. The double balloon is in a deflated state. The method further comprises placing the first balloon on a distal side of an anatomical opening in the ventricular space; placing the second balloon on a proximal side of the anatomical opening; aligning the second balloon with a target structure in the ventricular space; inflating the first balloon to stabilize the double balloon; after inflating the first balloon, inflating the second balloon to apply pressure to the target structure.

In one or more embodiments, the anatomical opening is an opening to Meckel's cave, and the target structure is a trigeminal ganglion. In one or more embodiments, the bridge includes a bridge balloon, and the method further comprises: aligning the bridge balloon with the anatomical opening; and inflating the bridge balloon to further stabilize the double balloon.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 is an illustration of the trigeminal ganglion.

FIG. 2 is a side view of an apparatus for treating trigeminal neuralgia according to an embodiment.

FIG. 3 is a side view of a distal portion of the apparatus illustrated in FIG. 2 to further illustrate the double balloon.

FIG. 4 is an enlarged view of the double balloon where the first balloon, the second balloon, and the bridge balloon are fluidly isolated from each other.

FIG. 5 is an enlarged view of the double balloon where the second balloon is larger than the first balloon according to another embodiment.

FIG. 6 is an enlarged view of the double balloon according to another embodiment.

FIG. 7 is a block diagram of a system for treating trigeminal neuralgia according to an embodiment.

FIG. 8 is a flow chart of a method for performing a medical procedure according to an embodiment.

FIG. 9 is a schematic diagram of the apparatus illustrated in FIG. 2 and/or the system illustrated in FIG. 7 as it performs certain steps of a medical procedure.

FIG. 10 is a schematic diagram of the apparatus illustrated in FIG. 2 and/or the system illustrated in FIG. 7 as it performs other steps of a medical procedure.

FIG. 11 is a side view of an apparatus for treating trigeminal neuralgia according to an alternative embodiment.

FIG. 12 is a block diagram of a system for treating trigeminal neuralgia according to an embodiment.

FIG. 13 is a flow chart of a method for performing a medical procedure according to another embodiment.

FIG. 14 is a schematic diagram of the apparatus illustrated in FIG. 11 and/or the system illustrated in FIG. 12 as it performs certain steps of a medical procedure.

FIG. 15 is a side view of an apparatus for treating trigeminal neuralgia according to another embodiment.

FIG. 16 is a block diagram of a system for treating trigeminal neuralgia according to another embodiment.

FIG. 17 is a flow chart of a method for performing a medical procedure according to another embodiment.

FIG. 18 is a schematic diagram of the apparatus illustrated in FIG. 15 and/or the system illustrated in FIG. 16 as it performs certain steps of a medical procedure.

FIGS. 19A and 19B are schematic diagrams of a cannula and a sharp stylet that can be used at the beginning of a medical procedure to access Meckel's cave.

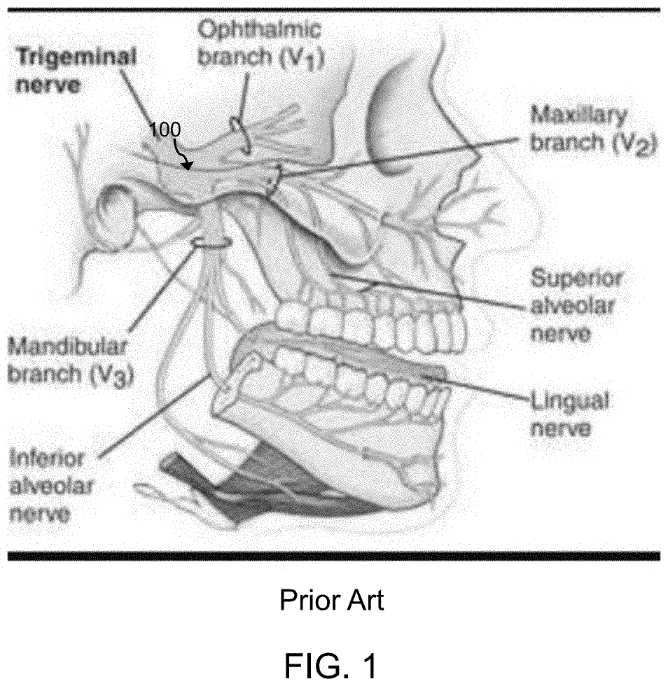

### DETAILED DESCRIPTION

An apparatus for treating trigeminal neuralgia includes a double balloon located at the distal end of a shaft. The double balloon includes a first balloon, a second balloon, and a bridge balloon that extends between the first and second balloons. The first balloon is located at the distal end of the apparatus. The bridge balloon extends from the proximal end of the first balloon to the distal end of the second balloon. The first balloon, the second balloon, and the bridge balloon can be inflated simultaneously or separately. The first and second balloons can have different sizes or diameters or they can have the same sizes or diameters. In a preferred embodiment, the first balloon has a larger diameter than the second balloon.

The first balloon and/or the bridge balloon, in the inflated state, can provide an anchor while the second balloon and/or the bridge balloon can be used to perform a therapeutic medical procedure on the trigeminal nerve. Additionally or alternatively, the first balloon and/or the bridge balloon in conjunction with the second balloon can at least partially block cerebral spinal fluid (CSF) flowing into or out of Meckel's cave, such as when a chemical therapeutic agent is used. For example, the first balloon can be disposed on the brain side of Meckel's cave, the bridge balloon can be disposed in the opening to Meckel's cave, and the second balloon be disposed in the entry to Meckel's cave.

In some embodiments, the apparatus can include an electrode arm that includes multiple electrical contacts to explore (e.g., by either stimulation and recording evoke potentials from the face or both) and locate a target trigeminal nerve that is responsible for the pain that the patient experiences from trigeminal neuralgia. The electrical contacts can be activated individually during exploration and therapeutic treatment (e.g., to perform RF ablative surgery). In addition or in the alternative, the apparatus can include an injectrode that includes a lumen through which a therapeutic agent can be delivered and one or more electrical contacts on the lumen. One or more electrical contact(s) on the injectrode can be used to explore and locate a target trigeminal nerve prior to injecting the therapeutic agent.

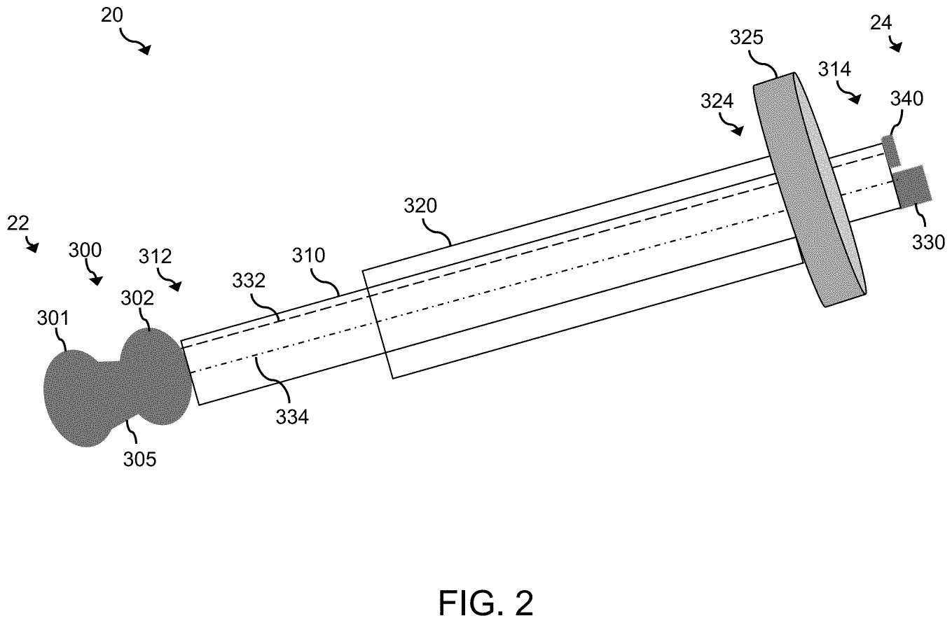

FIG. 2 is a side view of an apparatus 20 for treating trigeminal neuralgia according to an embodiment. The apparatus 20 includes a double balloon 300 disposed at a distal end 312 of a shaft 310 and/or at a distal end 22 of the apparatus 20. The shaft 310 can be rigid or flexible. In an embodiment, the shaft 310 is flexible in one or more directions to provide one or more degrees of freedom in which to orient the shaft 310. In some embodiments, one or more wires 332 can be disposed on and/or in the shaft 310 to direct the shaft 310 and/or the double balloon 300, such as during insertion and/or during a medical procedure. The wire(s) 332 can be mechanically coupled to a steering mechanism 340 at the proximal end 314 of the shaft 310 and/or at the proximal end 24 of the apparatus 20.

The double balloon 300 includes a first balloon 301, a second balloon 302, and a bridge balloon 305. The bridge balloon 305 can be replaced with an uninflatable bridge or bridge body in some embodiments. A distal end of one or more fluid lines 334 is fluidly coupled to the double balloon 300. The fluid line(s) can be fluidly coupled to the first balloon 301, to the second balloon 302, and/or to the bridge balloon 305. The fluid line(s) 334 are configured to change the inflation state of the double balloon 300. The fluid line(s) 334 can be located on or in the shaft 310. The double balloon 300 is in a deflated state in FIG. 2.

The fluid line(s) 334 can deliver fluid to the double balloon 300 to inflate the first balloon 301, the second balloon 302, and/or the bridge balloon 305. In addition, the fluid line(s) 334 can receive fluid from the double balloon 300 to deflate the first balloon 301, the second balloon 302, and/or the bridge balloon 305. The fluid for inflating and deflating the double balloon 300 can comprise a gas (e.g., air or another gas) or a liquid (e.g., saline, semi-saline, or another liquid).

A Luer lock 330 or other fluid coupling is disposed at a proximal end 314 of the shaft 310 and/or at a proximal end 24 of the apparatus 20. The Luer lock 330 is fluidly coupled to a proximal end of the fluid line(s) 334.

At least some of the shaft 310 can pass through an optional cannula 320 or other device to guide the shaft 310 and double balloon 320 during insertion into a patient. A proximal end 324 of the cannula 320 can include an optional handle 325. The optional handle 325 can include a locking mechanism to hold the shaft 310.

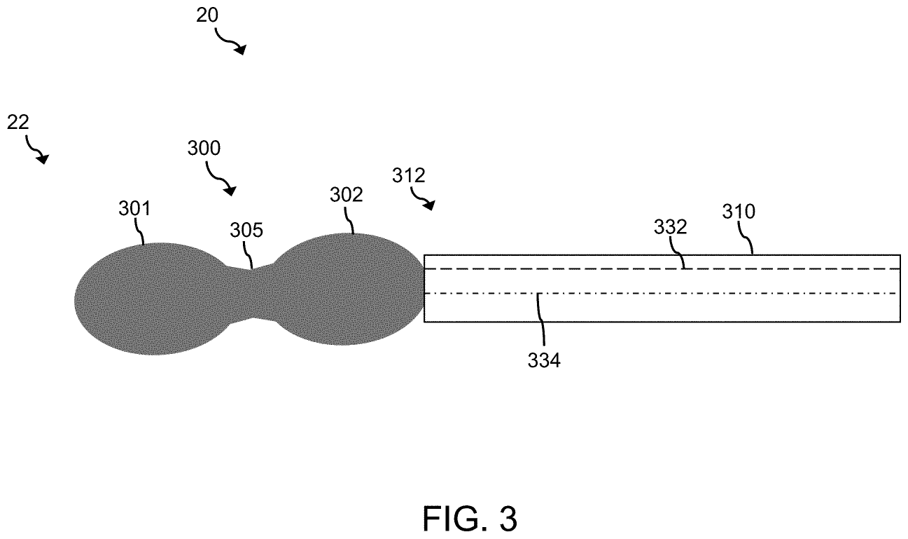

FIG. 3 is a side view of a distal portion of apparatus 20 to further illustrate the double balloon 300. The first balloon 301 is disposed at the distal end 22 of the apparatus 20. The second balloon 302 is disposed adjacent to the first balloon 301 at the distal end 312 of the shaft 310. The bridge balloon 305 extends between the first and second balloons 301, 302. For example, the bridge balloon 305 extends from the distal end of the second balloon 302 to the proximal end of the first balloon 301. The first balloon 301, the second balloon 302, and the bridge balloon 305 have a deflated state and an inflated state. In an alternative embodiment, the bridge balloon 305 cannot be inflated in which case only the first and second balloons 301, 302 have deflated and inflated states. The first balloon 301, the second balloon 302, and the bridge balloon 305 are in the deflated state in FIG. 3.

In one embodiment, the first balloon 301, the second balloon 302, and the bridge balloon 305 are fluidly coupled to (e.g., in fluid communication with) each other such that they inflate and deflate simultaneously. For example, the internal cavities of the first balloon 301, the second balloon 302, and the bridge balloon 305 can be open to allow fluid to flow into or out of the first balloon 301, the second balloon 302, and the bridge balloon 305 simultaneously. Additionally or alternatively, one or more fluid communication lines can extend between the first balloon 301, the second balloon 302, and the bridge balloon 305 to equalize the internal pressure in the first balloon 301, the second balloon 302, and the bridge balloon 305 which can cause the first balloon 301, the second balloon 302, and the bridge balloon 305 to inflate simultaneously and to deflate simultaneously.

In another embodiment, the first balloon 301, the second balloon 302, and/or the bridge balloon 305 is/are fluidly isolated from each other such that at least the first balloon 301, the second balloon 302, and/or the bridge balloon 305 can inflate and deflate independently of the other balloons. In a preferred embodiment, the first balloon 301, the second balloon 302, and the bridge balloon 305 are each fluidly isolated from each other so that the inflation state of each can be independently controlled. For example, the first balloon 301, the second balloon 302, and the bridge balloon 305 can be separately formed and/or can include a wall (e.g., an internal wall and/or an external wall) that separates each internal cavity from the neighboring internal cavity(ies).

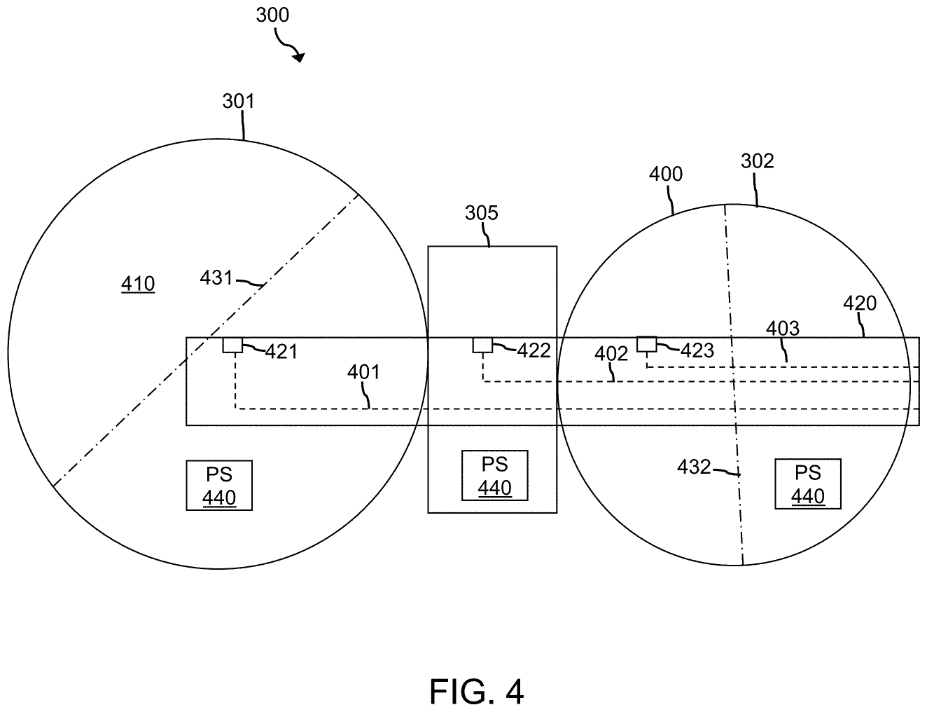

FIG. 4 is an enlarged view of the double balloon 300 where the first balloon 301, the second balloon 302, and the bridge balloon 305 are fluidly isolated from each other. Each of the first balloon 301, the second balloon 302, and the bridge balloon 305 is formed by a respective wall 400. The walls 400 define and separate the respective internal cavities 410 of the first balloon 301, the second balloon 302, and the bridge balloon 305. The first balloon 301, the second balloon 302, and the bridge balloon 305 can be separately inflated and deflated using dedicated fluid lines 401-403, respectively. The fluid lines 401-403 can be the same as or fluidly coupled to fluid line(s) 334. The fluid lines 401-403 can be disposed in an optional fluid coupling tube 420 that can include ports or apertures 421-423 that allow fluid to enter and exit the fluid lines 401-403, respectively. The fluid coupling tube 420 can extend to the proximal end 314 of the shaft 310 to fluidly couple the fluid lines 401-403 (e.g., using Luer lock 330 or similar fitting) with an external fluid reservoir, a pump, and/or a vacuum. The fluid can comprise a gas (e.g., air or another gas) or a liquid (e.g., saline, semi-saline, or another liquid), as discussed above.

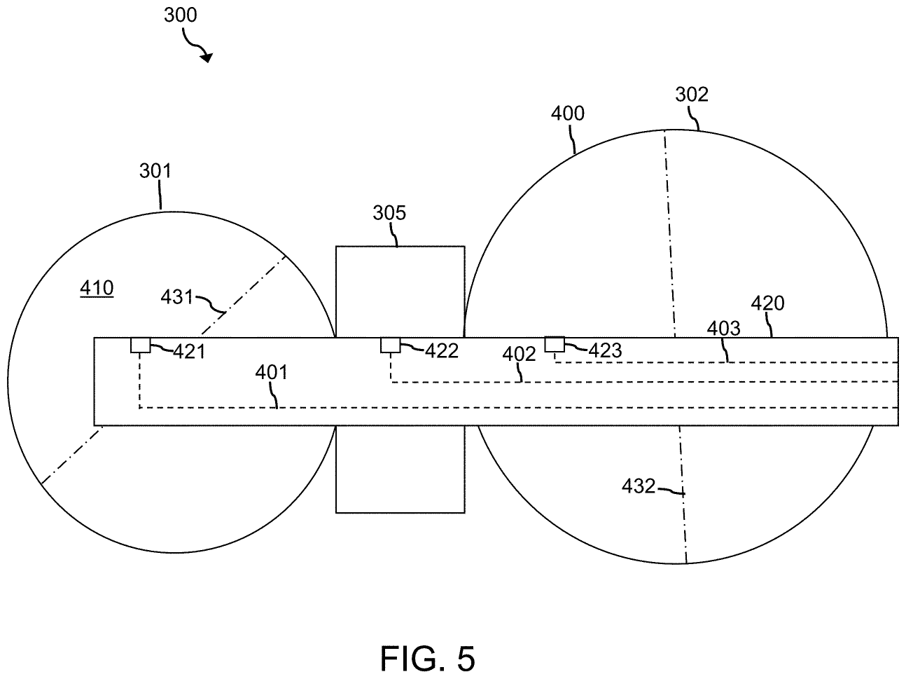

The first balloon 301, the second balloon 302, and the bridge balloon 305 are in the inflated state in FIG. 4. The first and second balloons 301, 302 can be the same size or can have different sizes. For example, a dimension of the first balloon 301 can be different than the corresponding dimension of the second balloon 302. In this figure, the first balloon 301 is larger in the inflated state than the second balloon 302 is in the inflated state. When the first and second balloons 301, 302 are spheres, a first dimension 431 (e.g., radius or diameter) of the first balloon 301 is greater than a corresponding second dimension 432 (e.g., radius or diameter) of the second balloon 302. In another embodiment, the second dimension 432 (e.g., radius or diameter) of the second balloon 302 is larger than the corresponding first dimension 431 (e.g., radius or diameter) of the first balloon 301, for example as illustrated in FIG. 5.

The double balloon 300 can be inflated using volume control or pressure control. In volume control, a predetermined volume of fluid can be added to the double balloon 300 (e.g., to the first balloon 301, to the second balloon 302, and/or to the bridge balloon 305). For example, a first predetermined volume of fluid can be added to the first balloon 301, a second predetermined volume of fluid can be added to the second balloon 302, and a third predetermined volume of fluid can be added to the bridge balloon 305. The predetermined volumes of fluid can be the same or different for each balloon 301, 302, 305. In addition, the predetermined volumes of fluid can be added simultaneously or separately (e.g., sequentially). The predetermined volumes of fluid can cause the first balloon 301, the second balloon 302, and the bridge balloon 305 to inflate regardless of the internal pressure or the external pressure or force caused by contact between the respective walls 400 and an anatomical surface.

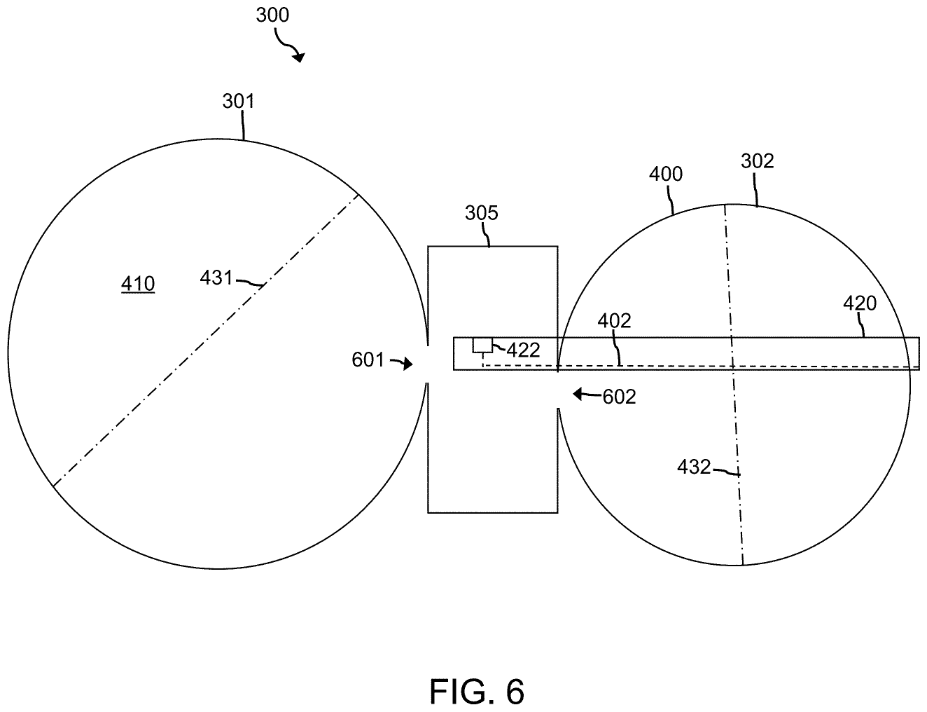

Alternatively, when the first balloon 301, the second balloon 302, and the bridge balloon 305 are fluidly coupled, a single predetermined volume of fluid can be used to inflate the first balloon 301, the second balloon 302, and the bridge balloon 305 simultaneously. For example, a single fluid line 422 can be fluidly coupled to the double balloon 300, such as to the bridge balloon 305, as illustrated in FIG. 6. The internal cavity 410 of the bridge balloon 305 is in fluid communication with the internal cavities 410 of the first and second balloons 301, 302 through apertures 601, 602, respectively, in the walls 400 of the first and second balloons 301, 302. In other embodiments, the single fluid line 422 can be fluidly coupled to the first balloon 301 or to the second balloon 302.

In pressure control, the double balloon 300 (e.g., the first balloon 301, the second balloon 302, and/or the bridge balloon 305) is inflated to a predetermined pressure. For example, when the first balloon 301, the second balloon 302, and the bridge balloon 305 are fluidly isolated from each other, the first balloon 301 can be inflated to a first predetermined pressure, the second balloon 302 can be inflated to a second predetermined pressure, and the bridge balloon 305 can be inflated to a third predetermined pressure. The predetermined pressures can be the same or different for each balloon 301, 302, 305. In addition, the first balloon 301, the second balloon 302, and/or the bridge balloon 305 can be inflated simultaneously or separately (e.g., sequentially). Alternatively, when the first balloon 301, the second balloon 302, and the bridge balloon 305 are fluidly coupled, they can be inflated simultaneously to a single predetermined pressure.

An optional internal pressure sensor 440 (FIG. 4) can be located in the first balloon 301, the second balloon 302, and/or the bridge balloon 305. The internal pressure sensor(s) 440 can be in wired or wireless communication with an external device (e.g., a computer, a smartphone, etc.) to provide an internal pressure measurement of the first balloon 301, the second balloon 302, and/or the bridge balloon 305. Additionally or alternatively, an external pressure sensor can be located externally from the balloons. The external pressure sensor can be in fluid communication with the first balloon 301, the second balloon 302, and/or the bridge balloon 305 to monitor the internal pressure in the first balloon 301, the second balloon 302, and/or the bridge balloon 305. For example, the external pressure sensor can be in fluid communication with fluid lines 401-403 and/or fluid line(s) 334.

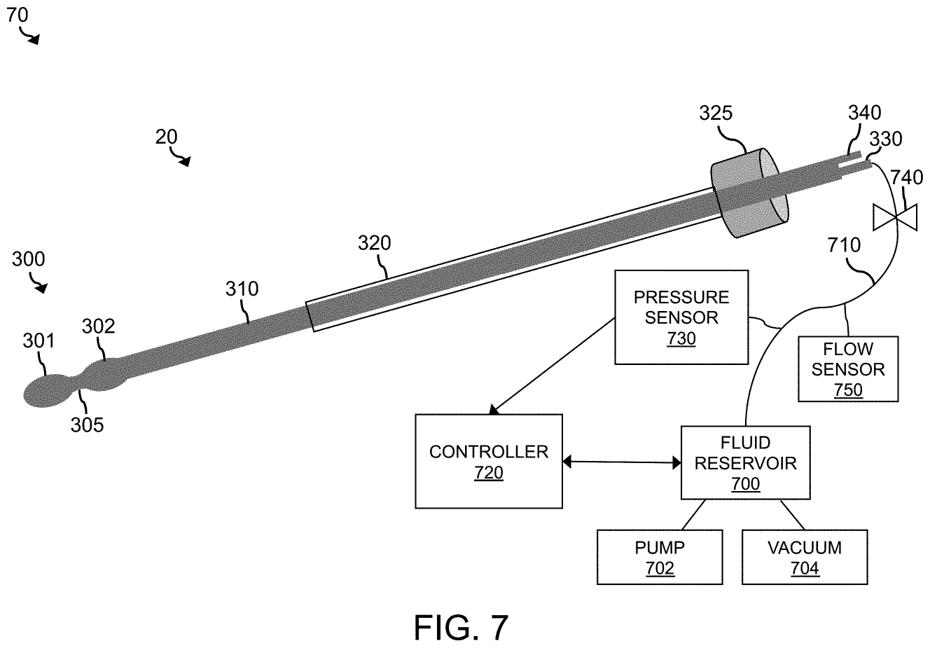

FIG. 7 is a block diagram of a system 70 for treating trigeminal neuralgia according to an embodiment. In system 70, the apparatus 20 is fluidly coupled to a fluid source or reservoir 700. To inflate some or all of the double balloon 300, such as the first balloon 301, the second balloon 302, and/or the bridge balloon 305, fluid is provided from the fluid source 700 to the relevant portion of the double balloon 300 via one or more fluid lines 710, which can be fluidly coupled to the fluid line(s) 334 (FIGS. 2, 3) and/or to the fluid line(s) 401-403 (FIGS. 4-6). A pump 702 can be fluidly coupled to the fluid source or reservoir 700 to drive the fluid into the relevant portion of the double balloon 300. To deflate some or all of the double balloon 300, such as the first balloon 301, the second balloon 302, and/or the bridge balloon 305, fluid is received in the fluid source or reservoir 700 from the relevant portion of the double balloon 300. A vacuum 704 can be fluidly coupled to the fluid source or reservoir 700 and/or to the fluid line(s) 334, 401-403 to suction the fluid out of the relevant portion of the double balloon 300. In some embodiments, the system 70 can include multiple fluid sources 700, pumps 702, and vacuums 704 to independently inflate and deflate different balloons in the double balloon 300.

Volume and/or pressure control inflation of the double balloon 300 can be controlled using an optional microprocessor-based controller 720. The controller 720 can be in electrical communication with the fluid source 700, the pump 702, and/or the vacuum 704 to cause the inflation and/or deflation of the double balloon 300. Additionally or alternatively, the controller 720 can be in electrical communication with one or more valves 740 in the fluid line(s) 710 to control the inflation and/or deflation of the double balloon 300.

An optional external pressure sensor 730 can be in fluid communication with the fluid line(s) 710 to measure the internal pressure of the fluid therein, which can correspond to the internal pressure of the first balloon 301, the internal pressure of the second balloon 302, and/or the internal pressure of the bridge balloon 305. When the fluid line(s) 710 include multiple fluid lines, multiple external pressure sensors 730 can be used to measure the internal pressure of the fluid in each fluid line 710. The output of the external pressure sensor(s) 730 and/or internal pressure sensor(s) 440 (FIG. 4) can be in electrical communication with the controller 720 which can automatically control the inflation (and/or deflation) of the first balloon 301, the second balloon 302, and/or the bridge balloon 305 based, at least in part, on the pressure sensed by the external pressure sensor(s) 730. The controller 720 can use the output of the pressure sensor(s) 440, 730 as feedback for example when inflating and/or deflating the first balloon 301, the second balloon 302, and/or the bridge balloon 305 using fluid source 700. For example, the controller 720 can activate or deactivate the pump 702 or vacuum 704 based on the output of the pressure sensor(s) 440, 730. Additionally or alternatively, the controller 720 can open or close the valve(s) 740 based on the output of the pressure sensor(s) 440, 730.

The controller 720 can control the inflation of the first balloon 301, the second balloon 302, and/or the bridge balloon 305 based on a target pressure and/or a maximum allowable pressure. The target pressure and the maximum allowable pressure can be specific for each of the first balloon 301, the second balloon 302, and/or the bridge balloon 305. Alternatively, when the first balloon 301, the second balloon 302, and/or the bridge balloon 305 are fluidly coupled, the target pressure and the maximum allowable pressure can be the same for the fluidly-coupled balloons, which have equalized pressures due to their fluid coupling.

An optional flow sensor 750 can be in fluid communication with the fluid line(s) 710 to measure the volumetric flow of fluid to and/or from the double balloon 300 during inflation and/or deflation, respectively. The output of the flow sensor 750 can be in electrical communication with the controller 720 which can automatically control the inflation (and/or deflation) of the first balloon 301, the second balloon 302, and/or the bridge balloon 305 based, at least in part, on the volumetric flow sensed by the flow sensor 750. When the fluid line(s) 710 include multiple fluid lines, multiple flow sensors 750 can be used to measure the volumetric flow in each fluid line 710. The controller 720 can control inflation and/or deflation based on a combination of volumetric control (e.g., using the output of the flow sensor(s) 750) and pressure control (e.g., using the output of the pressure sensor(s) 440, 730). For example, some or all of the double balloon 300 can be inflated using volumetric control provided that a maximum pressure is not reached. Alternatively, some or all of the double balloon 300 can be inflated using pressure control provided that a maximum volume has not been reached.

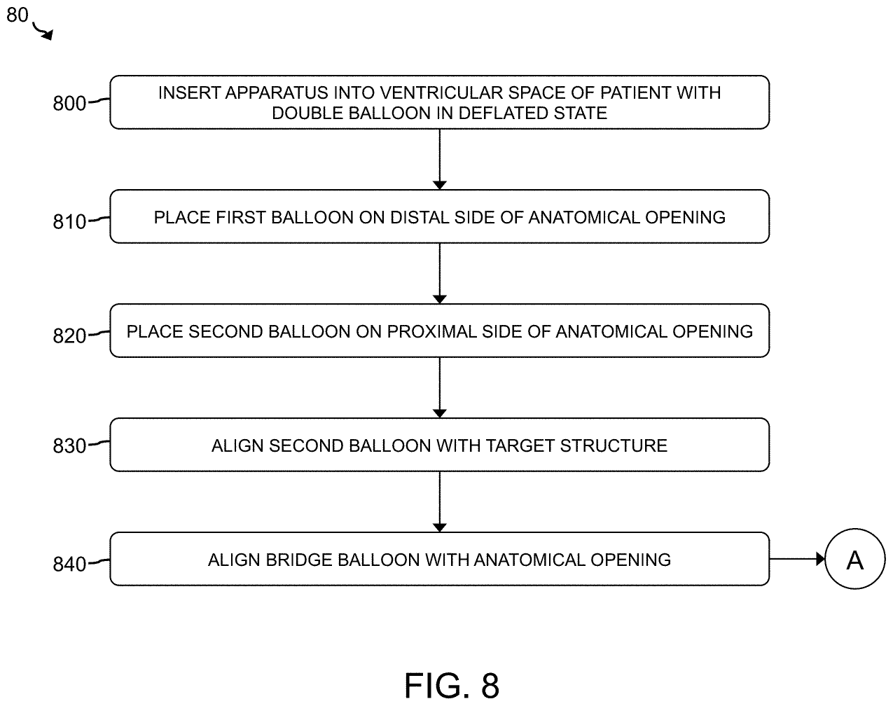

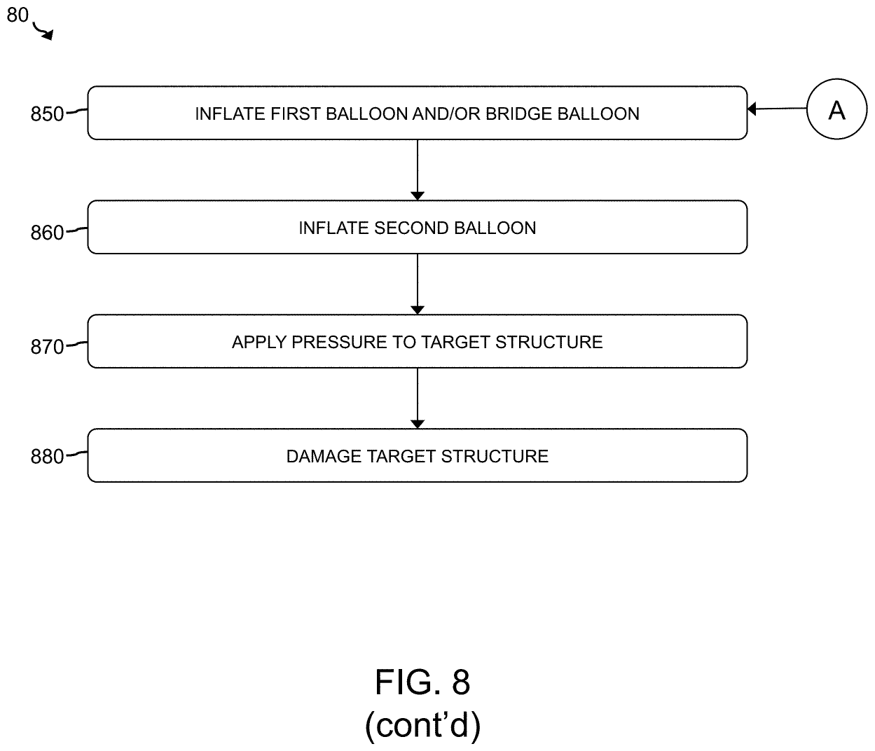

FIG. 8 is a flow chart of a method 80 for performing a medical procedure according to an embodiment. Method 80 can be performed using system 70. In step 800, the apparatus 20 is inserted into a ventricular space of a patient while the double balloon 300 is in a deflated state. An example of a ventricular space is the middle cranial fossa in which Meckel's cave (a/k/a the trigeminal cave, the trigeminal cavity, Meckel's cavity) is located.

In step 810, the first balloon 301 is placed on a distal side of an anatomical opening in the ventricular space. The anatomical opening can be an anatomical cavity, an anatomical recess (e.g., a dural recess), or another anatomical opening. In an embodiment, the anatomical opening is the opening to Meckel's cave. The distal side of the opening to Meckel's cave can include the brain space behind Meckel's cave. The first balloon 301 can be in the deflated state during step 810.

In step 820, the second balloon 302 is placed on a proximal side of the anatomical opening in the ventricular space. For example, the second balloon 302 can be placed on the proximal side of the opening to Meckel's cave (i.e., in Meckel's cave). The second balloon 302 can be in the deflated state during step 820.

In step 830, the second balloon 302 is aligned with a target structure. The target structure can be a nerve, a nerve bundle, a tumor, or other structure. In an embodiment, the target structure is or includes the trigeminal ganglion 100. The second balloon 302 can be in the deflated state during step 830.

In step 840, the bridge balloon 305 is aligned with the anatomical opening. For example, the bridge balloon 305 can be aligned with the opening to Meckel's cave. The bridge balloon 305 can be in the deflated state during step 840. Any of the balloons 301, 302, and/or 305 can be aligned using imaging or methods as known in the art.

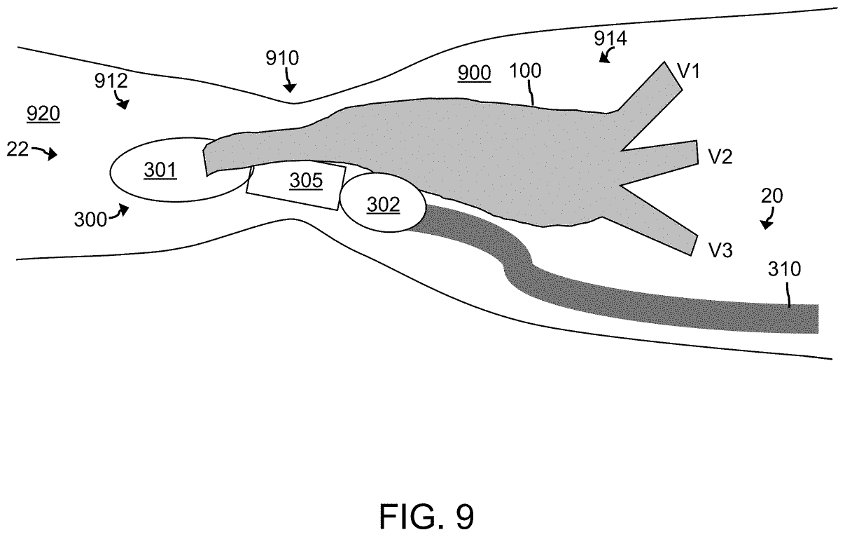

FIG. 9 is a schematic diagram of the apparatus 20 and/or system 70 as it performs steps 800-840 of method 80. The distal end 22 of the apparatus 30 is inserted through Meckel's cave 900 and past the trigeminal ganglion 100. The double balloon 300 is in the deflated state as it placed such that the first balloon 301 is located on the distal side 912 of the opening 910 to Meckel's cave 900. The distal side 912 of the opening 910 to Meckel's cave 900 includes the brain space 920 behind the opening 910 to Meckel's cave 900.

The bridge balloon 305 is located in and aligned with the opening 910 to Meckel's cave 900. The second balloon 302 is located on the proximal side 914 of the opening 910 to Meckel's cave 900 and is aligned with the trigeminal ganglion 100. The second balloon 302 can be located above, below, next to, or otherwise adjacent to the trigeminal ganglion 100. In an alternative embodiment, the bridge balloon 305 and/or the first balloon 301 can be aligned with the trigeminal ganglion 100.

In step 850 (via placeholder A), the first balloon 301 and/or the bridge balloon 305 is/are inflated. The first balloon 301 and/or the bridge balloon 305 can be inflated using pressure control, volume control, or a combination thereof, as discussed above. The first balloon 301 and/or the bridge balloon 305 can be inflated using any of the fluids discussed herein. When both the first balloon 301 and the bridge balloon 305 are inflated, the first balloon 301 and the bridge balloon 305 can be inflated simultaneously or sequentially. When both the first balloon 301 and the bridge balloon 305 are inflated sequentially, the first balloon 301 can be inflated before or after the bridge balloon 305. The first balloon 301 is preferably inflated before the bridge balloon 305 to secure the distal end of the double balloon 300.

In the inflated state, the first balloon 301 and/or the bridge balloon 305 can stabilize and/or anchor the double balloon 300 in the anatomical opening in the ventricular space. For example, the first balloon 301 and/or the bridge balloon 305 can stabilize and/or anchor the double balloon 300 in and/or around the opening to Meckel's cave.

In step 860, the second balloon 302 is inflated. The second balloon 302 is preferably inflated after the first balloon 301 and/or the bridge balloon 305 is/are inflated in step 850. The second balloon 302 can be inflated using pressure control, volume control, or a combination thereof, as discussed above. The second balloon 302 can be inflated using any of the fluids discussed herein.

In step 870, pressure or force is applied to the target structure by the inflated second balloon 302. The pressure or force causes damage to at least some of the target structure in step 880. For example, when the target structure is the trigeminal ganglion 100, the pressure or force from the second balloon 302 can damage at least some of the trigeminal ganglion 100, which can reduce at least some of the pain caused by a neurological condition such as trigeminal neuralgia.

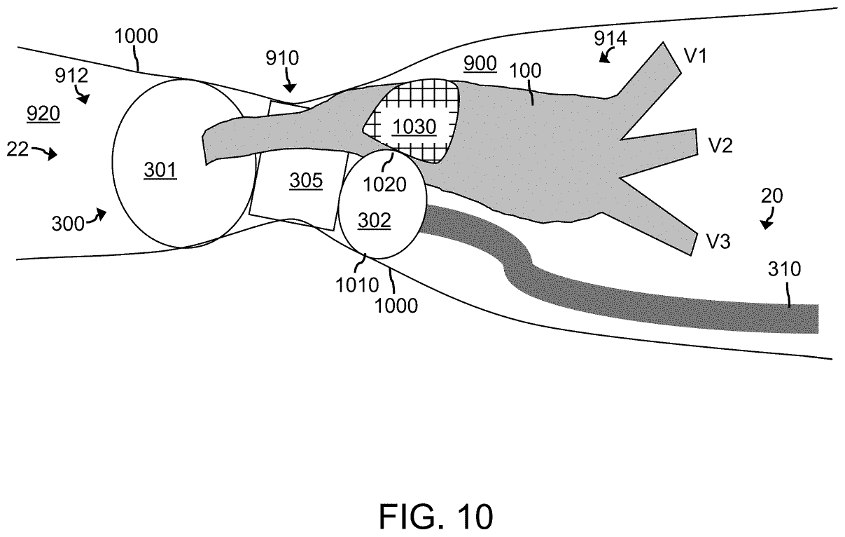

FIG. 10 is a schematic diagram of the apparatus 20 and/or system 70 as it performs steps 850-870 of method 80. In FIG. 10, the first balloon 301, the second balloon 302, and the bridge balloon 305 are inflated (e.g., in the inflated state). The first balloon 301 can secure the distal end of the double balloon 300, for example by pressing on an anatomical feature or wall 1000. The bridge balloon 305 can secure the middle of the double balloon 300 to the opening 910 to Meckel's cave, such as to the anatomical wall 1000 that defines the opening 910 to Meckel's cave.

The first balloon 301 and the bridge balloon 305 can be inflated to a respective predetermined volume and/or pressure. The predetermined pressure of the first balloon 301 and/or of the bridge balloon 305 can correspond to the restriction in volume expansion of the first balloon 301 and/or of the bridge balloon 305 when the first balloon 301 and/or the bridge balloon 305 contacts the anatomical wall 1000. Inflating the first balloon 301 and/or the bridge balloon 305 to a respective predetermined pressure can allow for consistent anchoring of the double balloon 300 during the procedure across patients that may have different size anatomical openings (e.g., different sizes of the opening 910 to Meckel's cave 900). Inflating the first balloon 301 and/or the bridge balloon 305 to a respective predetermined pressure can also allow for at least partial blocking of CSF during the procedure across patients that may have different size anatomical openings (e.g., different sizes of the opening 910 to Meckel's cave 900).

In the inflated state, the second balloon 302 can produce pressure or force on the trigeminal ganglion 100. For example, a first side 1010 of the second balloon 302 can contact an anatomical wall 1000 that defines Meckel's cave 900. This contact can cause a second side 1020 of the second balloon 302 to produce pressure or force on the trigeminal ganglion 100. The pressure or force can damage at least a portion 1030 of the trigeminal ganglion 100, which can reduce at least some of the pain caused by a neurological condition such as trigeminal neuralgia. When the second balloon 302 is inflated using pressure control, the predetermined pressure of the second balloon 302 can correspond to magnitude of force or pressure that is applied to the trigeminal ganglion 100.

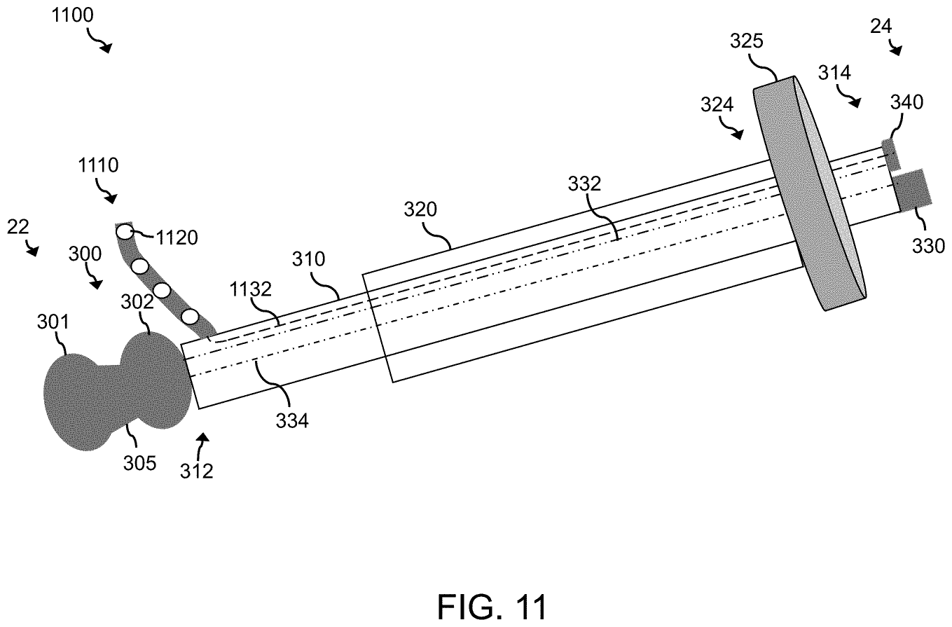

FIG. 11 is a side view of an apparatus 1100 for treating trigeminal neuralgia according to an alternative embodiment. The apparatus 1100 is the same as apparatus 20 except that apparatus 1100 includes an electrode arm 1110. The electrode arm 1110 includes a plurality of electrical contacts 1120 (e.g., electrodes) that are disposed along the length of the electrode arm 1110. The electrical contacts 1120 are preferably evenly spaced along the length of the electrode arm 1110. In other embodiments, the electrical contacts 1120 can be unevenly spaced along the length of the electrode arm 1110. In yet other embodiments, some of the electrical contacts 1120 can be evenly spaced and some of the electrical contacts 1120 can be unevenly spaced along the length of the electrode arm 1110.

The electrical contacts 1120 can be individually activated to provide electrical stimulation at different points along the length of the electrode arm 1110, which correspond to different trigeminal nerve locations. In a first example, one of the electrical contacts 1120 can be stimulated and a movement response may or may not be seen in the face. The production or inability to produce facial movements with electrical stimulation can provide information with regard to the location of the electrode arm 1110 and more generally of the double balloon 300 and apparatus 1100 with respect to the trigeminal ganglion 100. In a second example, electrical pads or needle electrodes can be placed on different locations on the patient's face that can correspond to facial regions that are connected to the V1-V3 branches of the trigeminal nerve. Test electrical stimulations can be sent through each of the electrode pads or needle electrodes on the face and the nerve responses can be monitored using the electrical contacts 1120. The target nerve branch can correspond to the trigeminal nerve branch responsible for sensation in the facial region(s) impacted by trigeminal neuralgia. After the target nerve branch is located, the operator can perform RF ablation therapy on the target nerve branch through the appropriate electrical contact(s) 1120 (e.g., the electrical contact(s) 1120 through which the test electrical stimulation was sent to determine the location of the target nerve branch). The double balloon 300 can be used to anchor apparatus 70 and/or provide structural stability for maneuvering the electrode arm 1110. Additionally or alternatively, the first balloon 301 can be inflated to apply pressure and/or force to damage at least a portion of the trigeminal ganglion 100 (e.g., in the same manner as discussed above).

In some embodiments, one or more wires 1132 can be coupled to the electrode arm 1110. The wire(s) 1132 can be used to steer the electrode arm 1110 independently of the shaft 310. For example, the shaft 310 can remain still while the wire(s) 1132 steer the electrode arm 1110. The wire(s) 1132 can be mechanically coupled to steering mechanism 340 or another steering mechanism at the proximal 24 end of apparatus 1100 and/or at the proximal end 314 of the shaft 310.

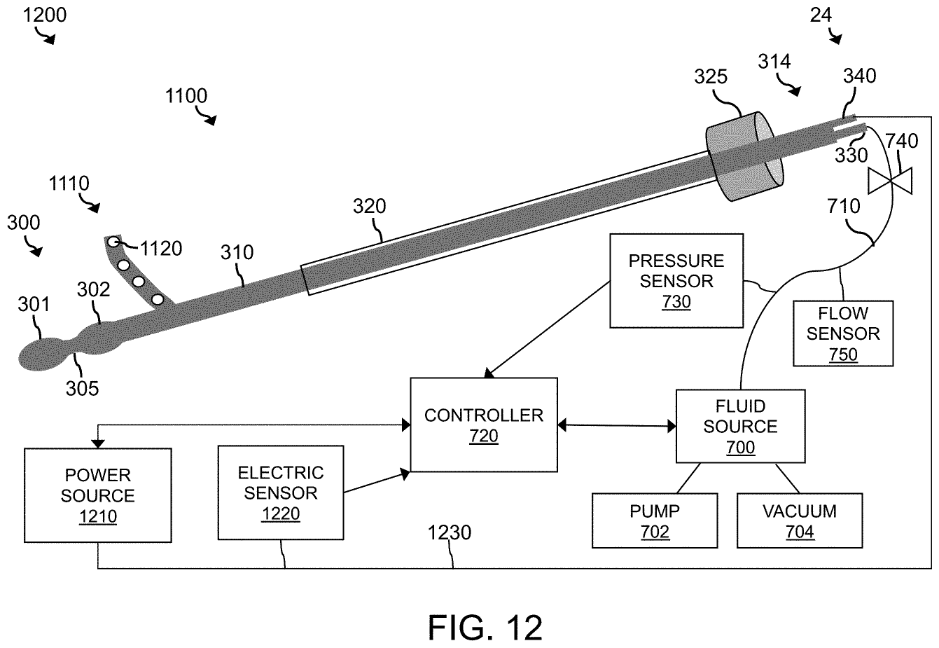

FIG. 12 is a block diagram of a system 1200 for treating trigeminal neuralgia according to another embodiment. System 1200 is the same as system 70 except that system 1200 includes apparatus 1100 instead of apparatus 20. In addition to the connections and controls described in system 70, system 1200 includes an electric power source 1210 that is electrically coupled to the electrical contacts 1120. The electric power source 1210 can provide a predetermined voltage and/or a predetermined current to the electrical contacts 1120. In some embodiments, a first voltage and/or current can be used to electrically stimulate the trigeminal ganglion 100 to determine the trigeminal nerve branch or location responsible for some or all of the pain caused by trigeminal neuralgia. After the target trigeminal nerve branch or location is located, a second current and/or voltage can be used to perform RF ablation therapy. The second current and/or voltage is higher than the first current and/or voltage. In addition, the second current and/or voltage can be at a higher AC frequency than the first current and/or voltage. In an embodiment, the first current and/or voltage is a DC current and/or a DC voltage, respectively.

One or more electric sensors 1220 can be electrically coupled to the wires 1230 that electrically couple the electric power source 1210 and the electrical contacts 1120. The electric sensor(s) 1220 can measure the current and/or voltage applied to the electric sensor(s) 1220, which can be used as feedback to the controller 720, which is in electrical communication with the electric power source 1210 (e.g., to provide control signals thereto). In addition, the electric sensor(s) 1220 can measure the current and/or voltage of any electrical signals sensed by the electrical contacts 1120 from any trigeminal nerve responses to test electrical stimulations through electrode pads or needle electrodes on the face to locate the target trigeminal nerve branch for therapy. The wires 1230 preferably form parallel electrical connections to the electrical contacts 1120 so that electrical signals and/or power can be sent and received to/from each electrical contact 1120 individually. The output of the electric sensor(s) 1220 is in electrical communication with the controller 720 to provide feedback to the controller 720.

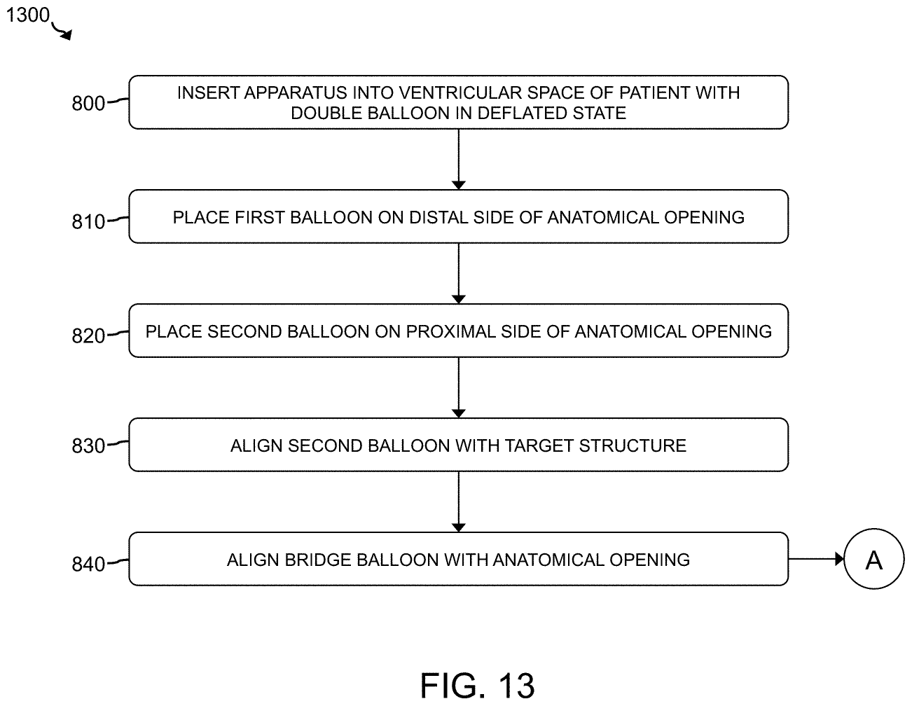

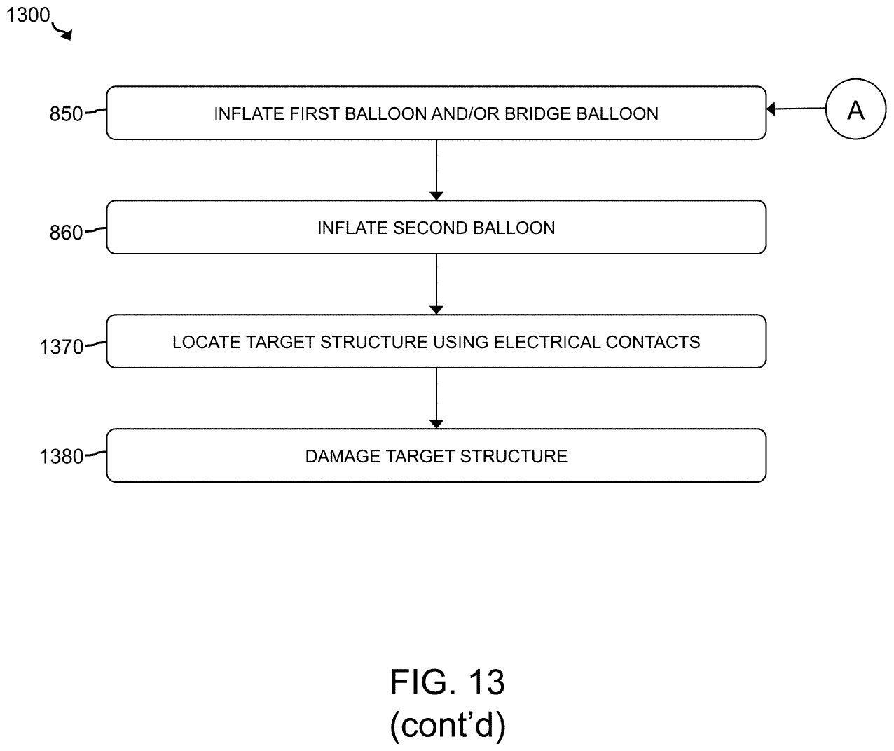

FIG. 13 is a flow chart of a method 1300 for performing a medical procedure according to another embodiment. Method 1300 can be performed using system 1200. Steps 800-860 of method 1300 are the same as described above with respect to method 80. When the second balloon is inflated in step 860, the second balloon preferably does not damage the target structure. To prevent damage, the second balloon used in method 1300 can be smaller than the second balloon used in method 80. Alternatively, the second balloon can be partially inflated in step 860 of method 1300.

In step 1370, the target structure is located using the electrical contacts 1120 in the electrode arm 1110. For example, the electrical contacts 1120 can be individually activated to provide electrical stimulation at different points along the length of the electrode arm 1110, which can correspond to different trigeminal nerve locations. The production or inability to produce facial movements with electrical stimulation from the electrical contacts 1120 can provide information with regard to the location of the electrode arm 1110 and more generally of the double balloon 300 and apparatus 1100 with respect to the target structure (e.g., the trigeminal ganglion 100). In a second example, electrical pads or needle electrodes can be placed on different locations on the patient's face that can correspond to facial regions that are connected to the V1-V3 branches of the trigeminal nerve. Test electrical stimulations can be sent through each of the electrode pads or needle electrodes on the face and the nerve responses can be monitored using the electrical contacts 1120 and electric sensor 1220. The target nerve branch can correspond to the trigeminal nerve branch responsible for sensation in the facial region(s) impacted by trigeminal neuralgia.

In step 1380, the target structure is damaged. The target structure can be damaged through RF ablation therapy using the appropriate electrical contact(s) 1120 (e.g., the electrical contact(s) 1120 through which the test electrical stimulation was sent to determine the location of the target nerve branch). The double balloon 300 can be used to anchor apparatus 70 and/or provide structural stability for maneuvering the electrode arm 1110. Additionally or alternatively, the first balloon 301 can be inflated to apply pressure and/or force to damage at least a portion of the trigeminal ganglion 100 (e.g., in the same manner as in step 880). The damage to the trigeminal ganglion can reduce the pain caused by trigeminal neuralgia.

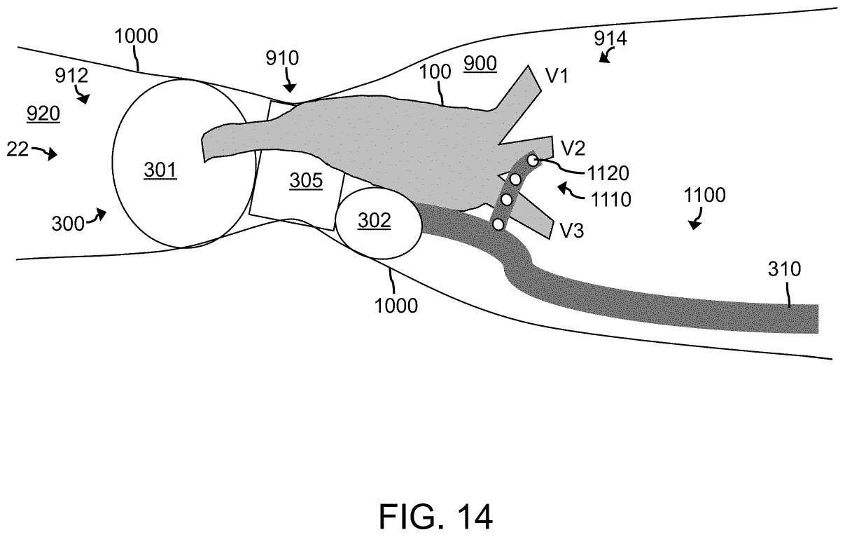

FIG. 14 is a schematic diagram of the apparatus 1100 as it performs steps 1370 and 1380 of method 1300. In FIG. 14, the electrode arm 1110 is pivoted such that it extends proximal to the V2 and V3 branches of the trigeminal ganglion 100. The electrical contacts 1120 can individually deliver electrical stimulation to and/or sense nerve responses that pass through the V2 and V3 branches of the trigeminal ganglion 100. The double balloon 300 is in the inflated state in FIG. 14.

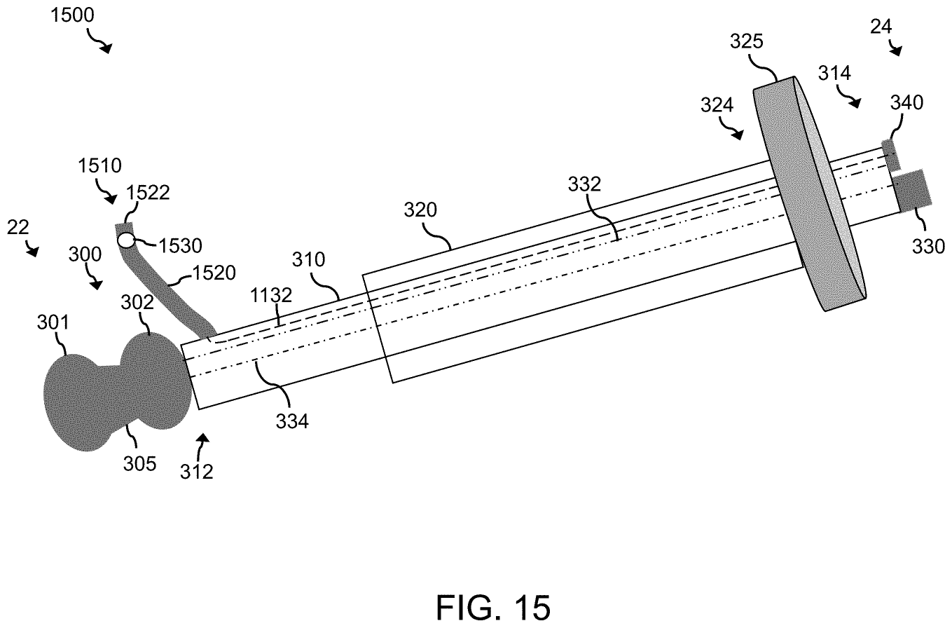

FIG. 15 is a side view of an apparatus 1500 for treating trigeminal neuralgia according to another embodiment. The apparatus 1500 is the same as apparatus 1100 except that apparatus 1500 includes an injectrode 1510 instead of an electrode arm 1110. The injectrode 1510 includes a lumen 1520 through which a therapeutic agent can be delivered and one or more electrical contacts 1530 (e.g., electrode(s)) disposed on the lumen 1520). The electrical contact(s) 1530 can be located near the distal open end 1522 of lumen 1520 and/or along the shaft of the lumen 1520.

A therapeutic agent can be delivered through the distal open end 1522 of lumen 1520. Electrical pads or needle electrodes can be placed on different locations on the patient's face that can correspond to facial regions that are connected to the V1-V3 branches of the trigeminal nerve. The electrical pads or needle electrodes can be individually activated to provide electrical stimulation and the nerve signal can be monitored using the contact(s) 1530 to locate a target trigeminal nerve location (e.g., one or more branches of the trigeminal nerve) that is responsible for sensation in the facial region(s) impacted by trigeminal neuralgia. In some embodiments, the injectrode 1510 can include multiple electrical contacts 1530, such as the electrical contacts 1120 in electrode arm 1110. Additionally or alternatively, test electrical stimulations can be sent through the electrical contact(s) 1530 individually while monitoring the patients face, for the production or lack of motor movements, as feedback to determine a target trigeminal nerve location.

After the target trigeminal nerve location is located, the operator can inject a therapeutic agent through the lumen 1520 so that the therapeutic agent is injected close to the target trigeminal nerve location. The double balloon 300 (e.g., the first balloon 301 and/or the bridge balloon 305) can at least partially block flow of CSF so that the therapeutic agent is not displaced from the target location through the flow of CSF or diluted by CSF, which can reduce the therapeutic effect of the therapeutic agent and/or can harm nerve locations that are not related to the patient's trigeminal neuralgia. In addition, the double balloon 300 can be used to anchor apparatus 1500 and/or provide structural stability for maneuvering the injectrode 1510.

In addition, the at least partial blockage of CSF can allow a relative high concentration of the therapeutic agent to be maintained at or near the target trigeminal nerve location. The therapeutic agent can include a chemical agent, a viral agent, and/or another therapeutic substance such as a genetic-altering (e.g., modulating) agent, a protein-altering (e.g., modulating) agent, and/or a cellular-altering (e.g., modulating) agent.

One or more wires 1132 can be coupled to the injectrode 1510 to steer the injectrode 1510 independently of the shaft 310. For example, the shaft 310 can remain still while the wire(s) steer the injectrode 1510. The wire(s) 1132 can be mechanically coupled to steering mechanism 340 or another steering mechanism at the proximal end 24 of the apparatus 1500.

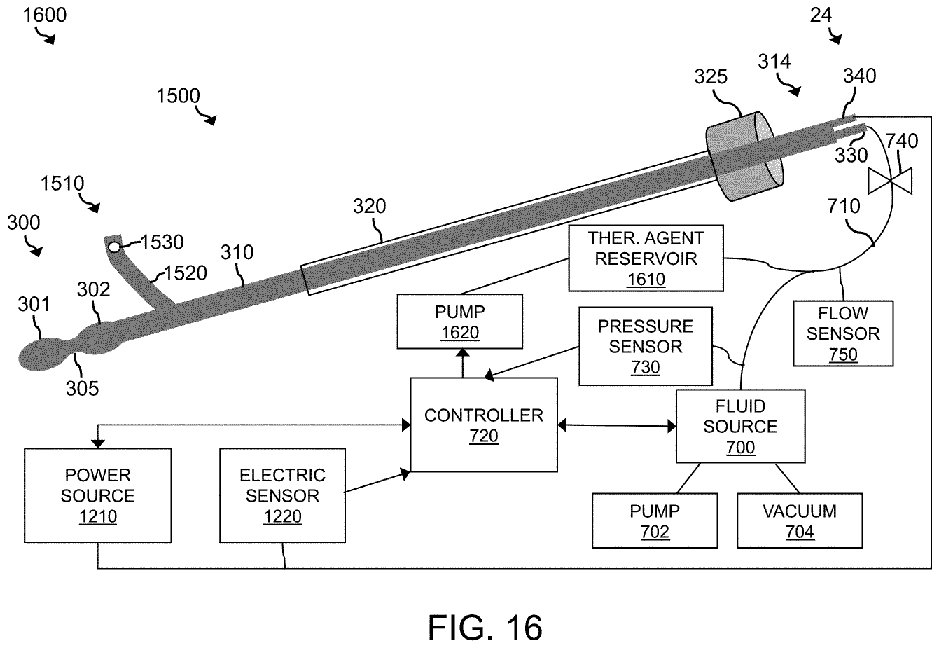

FIG. 16 is a schematic diagram of a system 1600 for treating trigeminal neuralgia according to an embodiment. System 1600 is the same as system 1200 except that system 1600 includes apparatus 1500 instead of apparatus 1100. In addition to the connections and controls described in system 1200, system 1600 includes a therapeutic agent reservoir 1610 and an optional pump 1620. The therapeutic agent reservoir 1610 is fluidly coupled to the injectrode 1510 (e.g., via fluid line(s) 710) to provide the therapeutic agent to the lumen 1520 of injectrode 1510. The optional pump 1620 can provide pressure to drive the therapeutic agent to flow into the lumen 1520. The therapeutic agent reservoir 1610 and/or the optional pump 1620 can be in electrical communication with the controller 720. The power source 1210 and electrical sensor 1220 can function in the same manner with respect to electrical contact(s) 1530 as described above with respect to electrical contacts 1120.

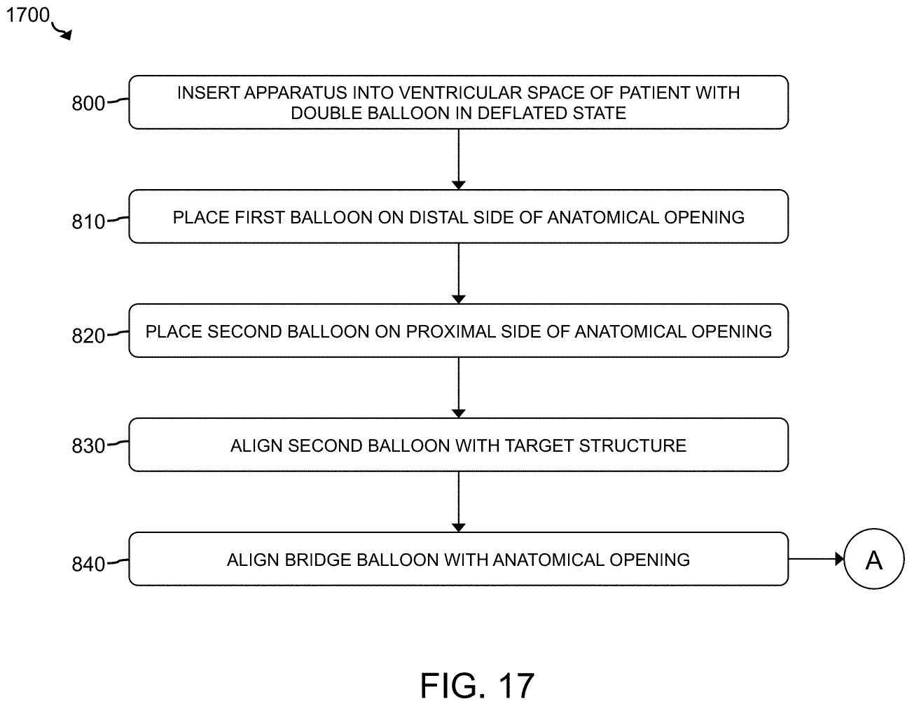

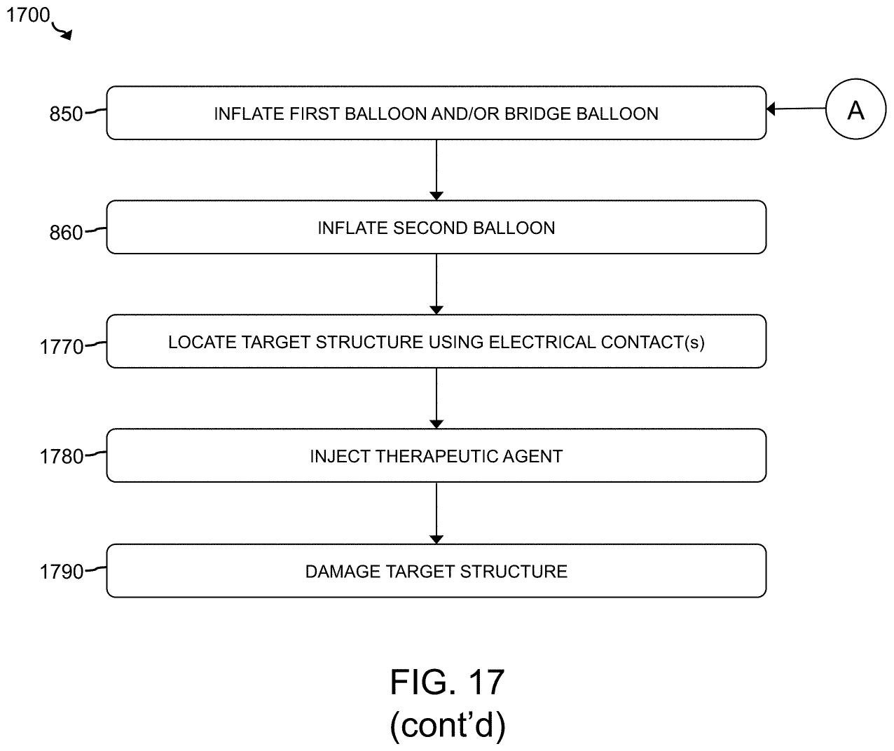

FIG. 17 is a flow chart of a method 1700 for performing a medical procedure according to another embodiment. Method 1700 can be performed using system 1600. Steps 800-860 of method 1700 are the same as described above with respect to method 1300.

In step 1770, the target structure is located using the electrical contact(s) on the injectrode 1510. For example, the electrical contact(s) 1530 can be individually activated to provide electrical stimulation at different points along the length of the lumen 1520, which can correspond to different trigeminal nerve locations or branches. The production or inability to produce facial movements with electrical stimulation from the electrical contacts 1530 can provide information with regard to the location of the injectrode 1510 and more generally of the double balloon 300 and apparatus 1500 with respect to the target structure (e.g., the trigeminal ganglion 100). In a second example, electrical pads or needle electrodes can be placed on different locations on the patient's face that can correspond to facial regions that are connected to the V1-V3 branches of the trigeminal nerve. Test electrical stimulations can be sent through each of the electrode pads or needle electrodes on the face and the nerve responses can be monitored using the electrical contacts 1120 and electric sensor 1220. The target nerve branch can correspond to the trigeminal nerve branch responsible for sensation in the facial region(s) impacted by trigeminal neuralgia. Step 1770 can be performed in the same manner as step 1370 in some embodiments.

In step 1780, a therapeutic agent is injected from the injectrode. The therapeutic agent is preferably injected towards or near the target structure (e.g., a target trigeminal nerve location or branch) located in step 1770. The therapeutic agent causes damage to at least a portion of the target structure in step 1790. The double balloon (e.g., the first balloon and/or the bridge balloon) can at least partially block CSF from flowing through the opening to Meckel's cave, which can allow the concentration of the therapeutic agent to remain relatively high and can prevent the therapeutic agent from damaging other nerves.

In step 1790, the target structure (e.g., a target trigeminal nerve location or branch) is damaged by the therapeutic agent. Additionally or alternatively, the first balloon can be further inflated to apply pressure and/or force to damage at least a portion of the target structure (e.g., in the same manner as in step 880). When the target structure includes the trigeminal ganglion, the damage to the trigeminal ganglion can reduce the pain caused by trigeminal neuralgia.

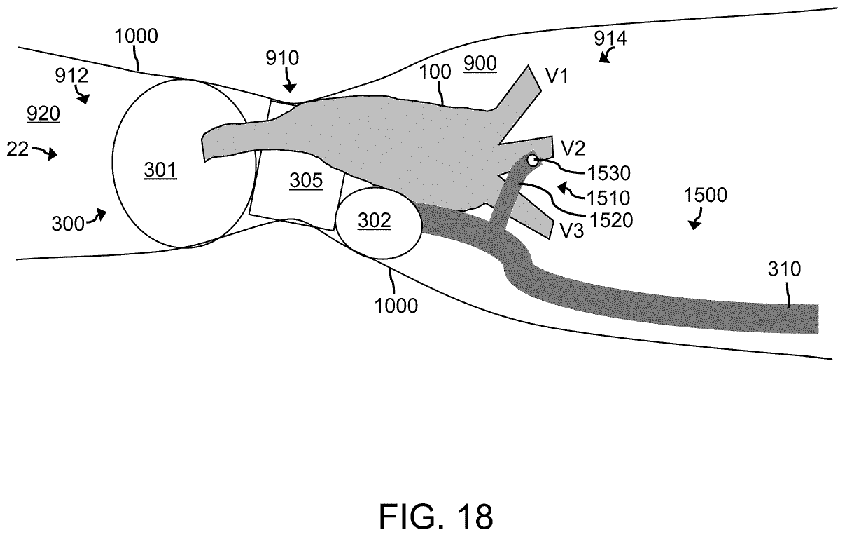

FIG. 18 is a schematic diagram of the apparatus 1500 and/or system 1600 as it performs steps 1770-1790 of method 1700. In FIG. 18, the injectrode 1510 is pivoted such that it extends proximal to the V2 and V3 branches of the trigeminal ganglion 100. The electrical contact(s) 1530 can individually deliver electrical stimulation to and/or sense nerve responses that pass through the V2 and V3 branches of the trigeminal ganglion 100 to determine if the V2 and/or V3 branch is the target structure. The injectrode 1510 can then inject a therapeutic agent proximal to the target structure (e.g., after optional repositioning of the injectrode 1510) to damage at least a portion of the target structure. The double balloon 300 is in the inflated state in FIG. 18.

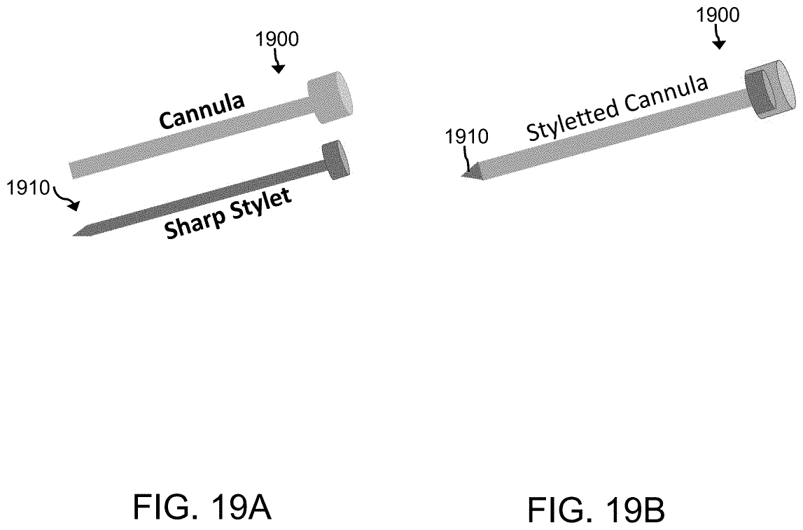

FIGS. 19A and 19B are schematic diagrams of a cannula 1900 and a sharp stylet 1910 that can be used at the beginning of a medical procedure to access Meckel's cave. When the distal end of the cannula reaches Meckel's cave, the sharp stylet is removed and apparatus 20, 1100, or 1500 is inserted to perform a therapeutic procedure, as discussed above. In some embodiments, a guidewire can be inserted and attached to apparatus 20, 1100, or 1500. FIG. 19A illustrates the cannula 1900 and sharp stylet 1910 separately. FIG. 19B illustrates the sharp stylet 1910 inserted into the cannula 1900. In addition, another probe can be inserted into cannula 1900 as a means of exploring or visualizing Meckel's cave. Cannula 1900 can be the same as cannula 320.

The invention should not be considered limited to the particular embodiments described above. Various modifications, equivalent processes, as well as numerous structures to which the invention may be applicable, will be readily apparent to those skilled in the art to which the invention is directed upon review of this disclosure. The above-described embodiments may be implemented in numerous ways. One or more aspects and embodiments involving the performance of processes or methods may utilize program instructions executable by a device (e.g., a computer, a processor, or other device) to perform, or control performance of, the processes or methods.

In this respect, various inventive concepts may be embodied as a non-transitory computer readable storage medium (or multiple non-transitory computer readable storage media) (e.g., a computer memory of any suitable type including transitory or non-transitory digital storage units, circuit configurations in Field Programmable Gate Arrays or other semiconductor devices, or other tangible computer storage medium) encoded with one or more programs that, when executed on one or more computers or other processors, perform methods that implement one or more of the various embodiments described above. When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the aspects described above. In some embodiments, computer readable media may be non-transitory media.

The terms “program,” “app,” and “software” are used herein in a generic sense to refer to any type of computer code or set of computer-executable instructions that may be employed to program a computer or other processor to implement various aspects as described above. Additionally, it should be appreciated that, according to one aspect, one or more computer programs that when executed perform methods of this application need not reside on a single computer or processor, but may be distributed in a modular fashion among a number of different computers or processors to implement various aspects of this application.

Computer-executable instructions may be in many forms, such as program modules, executed by one or more computers or other devices. Generally, program modules include routines, programs, objects, components, data structures, etc. that performs particular tasks or implement particular abstract data types. The functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Thus, the disclosure and claims include new and novel improvements to existing methods and technologies, which were not previously known nor implemented to achieve the useful results described above. Users of the method and system will reap tangible benefits from the functions now made possible on account of the specific modifications described herein causing the effects in the system and its outputs to its users. It is expected that significantly improved operations can be achieved upon implementation of the claimed invention, using the technical components recited herein.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. An apparatus comprising:
a shaft having proximal and distal ends;
a double balloon attached to the distal end of the shaft, the double balloon comprising:
a first balloon;
a second balloon; and
a bridge extending between the first and second balloons; and
one or more fluid lines disposed in the shaft, the fluid line(s) fluidly coupled to the double balloon and configured to change an inflation state of the double balloon.

2. The apparatus of claim 1, wherein the one or more fluid lines include:
a first fluid line fluidly coupled to the first balloon, and
a second fluid line fluidly coupled to the second balloon.

3. The apparatus of claim 2, wherein the first balloon and the second balloon are fluidly isolated from each other, whereby an inflation state of the first balloon is independent from an inflation state of the second balloon.

4. The apparatus of claim 3, wherein:
the first balloon has a first cavity to receive fluid from the first fluid line,
the second balloon has a second cavity to receive fluid from the second fluid line,
and the apparatus further comprises:
a first pressure sensor in fluid communication with first cavity; and
a second pressure sensor in fluid communication with second cavity.

5. The apparatus of claim 3, wherein:
the bridge comprises a bridge balloon having a bridge inflation state, and
the bridge balloon is fluidly coupled to the first balloon or the second balloon, whereby:
when the bridge balloon is fluidly coupled to the first balloon, the bridge inflation state is the same as the first inflation state, and
when the bridge balloon is fluidly coupled to the second balloon, the bridge inflation state is the same as the second inflation state.

6. The apparatus of claim 3, wherein:
the bridge comprises a bridge balloon,
the one or more fluid lines include a third fluid line fluidly coupled to the bridge balloon,
the first balloon, the second balloon, and the third balloon are fluidly isolated from each other,
whereby an inflation state of the first balloon, an inflation state of the second balloon, and an inflation state of the bridge balloon are independent from each other.

7. The apparatus of claim 1, wherein when the first and second balloons are inflated, a first dimension of the first balloon is larger than a corresponding second dimension of the second balloon.

8. The apparatus of claim 7, wherein when the first and second balloons are inflated, the first balloon has a larger diameter than the second balloon.

9. The apparatus of claim 7, wherein the second balloon is located between the bridge and the shaft.

10. The apparatus of claim 1, further comprising an electrode contact arm attached to the shaft, the electrode arm including a plurality of electrical contacts.

11. The apparatus of claim 10, further comprising one or more wires mechanically coupled to the electrode arm to steer the electrode arm independently of the shaft.

12. The apparatus of claim 1, further comprising an injectrode attached to the shaft.

13. The apparatus of claim 12, wherein the injectrode includes:
a lumen to deliver a therapeutic agent; and
one or more electrodes disposed on the lumen.

14. The apparatus of claim 13, further comprising one or more wires mechanically coupled to the lumen to steer the injectrode independently of the shaft.

15. A system comprising:
a shaft having proximal and distal ends;
a double balloon attached to the distal end of the shaft, the double balloon comprising:
a first balloon;
a second balloon; and
a bridge extending between the first and second balloons;
one or more fluid lines disposed in the shaft, the fluid line(s) fluidly coupled to the double balloon and configured to change an inflation state of the double balloon; and
a fluid reservoir fluidly coupled to the one or more fluid lines.

16. The system of claim 15, further comprising a pressure sensor configured to measure a pressure of the one or more fluid lines.

17. The system of claim 16, further comprising:
a pump fluidly coupled to the fluid reservoir; and
a controller in electrical communication with the pressure sensor and the pump, the controller configured to control the pump to inflate the first and second balloons while using the pressure of the one or more fluid lines as feedback.

18. A method of performing a therapeutic procedure, comprising:
inserting an apparatus into a ventricular space of a patient, the apparatus including:
a shaft having proximal and distal ends;
a double balloon attached to the distal end of the shaft, the double balloon comprising:
a first balloon;
a second balloon; and
a bridge extending between the first and second balloons; and
one or more fluid lines disposed in the shaft, the fluid line(s) fluidly coupled to the double balloon and configured to change an inflation state of the double balloon,
wherein the double balloon is in a deflated state;
placing the first balloon on a distal side of an anatomical opening in the ventricular space;
placing the second balloon on a proximal side of the anatomical opening;
aligning the second balloon with a target structure in the ventricular space;
inflating the first balloon to stabilize the double balloon;
after inflating the first balloon, inflating the second balloon to apply pressure to the target structure.

19. The method of claim 18, wherein:
the anatomical opening is an opening to Meckel's cave, and
the target structure is a trigeminal ganglion.

20. The method of claim 18, wherein:
the bridge includes a bridge balloon, and
the method further comprises:
aligning the bridge balloon with the anatomical opening; and
inflating the bridge balloon to further stabilize the double balloon.
